(19) United States
(12) Patent Application Publication
(10) Pub. No.: US 2026/0232390 A1
(43) Pub. Date: Aug. 13, 2026

(54) ROBOTIC SURGICAL SYSTEM WITH BEDSIDE ASSIST AND RELATED METHODS

(71) Applicant: Cilag GmbH International, Zug (CH)

(72) Inventors: Andrew T. Beckman, Cincinnati, OH (US); Evan N. Stambler, West Chester, OH (US)

(21) Appl. No.: 19/049,227

(22) Filed: Feb. 10, 2025

(51) Int. Cl. A61B 34/30; A61B 17/34; A61B 46/10

(52) U.S. Cl. CPC A61B 34/30; A61B 46/10; A61B 17/3476

(57) ABSTRACT

A robotic surgical system, comprising: a robotic arm including a distal end; a tool driver operatively coupled with the distal end of the robotic arm and including a carriage at a proximal end; a control unit; and an instrument adapter comprising: a carriage mount portion, and a secondary tool clamp portion, wherein the carriage mount portion is operatively receivable on the carriage at the proximal end of the tool driver, and wherein the secondary tool clamp portion is operatively configured to provide resistance and hold a secondary tool in position.

### BACKGROUND

A variety of medical instruments may be used in procedures conducted by a medical professional operator, as well as applications in robotically assisted surgeries. In the case of robotically assisted surgery, the clinician may operate a master controller to remotely control the motion of such medical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more input devices (such as foot pedals, joysticks, exoskeletol gloves, master manipulators, or the like), which are coupled by a servo mechanism to the medical instrument. In some scenarios, a servo motor moves a manipulator supporting the medical instrument based on the clinician's manipulation of the hand input devices. During the medical procedure, the clinician may employ, via a robotic system, a variety of medical instruments including an ultrasonic blade, a surgical stapler, a tissue grasper, a needle driver, an electrosurgical cautery probes, etc. Each of these structures performs functions for the clinician, for example, cutting tissue, coagulating tissue, holding, or driving a needle, grasping a blood vessel, dissecting tissue, or cauterizing tissue.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; and U.S. Pat. No. 11,058,493, entitled “Robotic System Configured for Navigation Path Tracing,” issued Jul. 13, 2021, the disclosure of which is incorporated by reference herein, in its entirety.

During one example of a medical procedure, the clinician may prefer to use one or more secondary devices during the procedure, but which does not need to be controlled via a robotic system, such as, but not limited to, scopes, liver retractors, and/or graspers. In these situations, the secondary devices will often be handled by an additional bedside clinician or assistant, which may further complicate staffing of such medical procedure and lead to increased cost to the patient.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a surgeon console and a plurality of robotic arms;

FIG. 2 depicts a perspective view of an example of a robotic arm, an example of a tool drive, and an example of a surgical instrument, each configured with use with the table-based robotic system of FIG. 1;

FIG. 3A depicts an enlarged schematic perspective view of the tool driver and surgical instrument of FIG. 2;

FIG. 3B depicts a schematic perspective view of the tool driver similar to FIG. 3A, but with the surgical instrument removed to expose rotary drives;

FIG. 4 depicts a schematic perspective view of one version of an instrument adapter of the present disclosure secured to a carriage of a tool driver

FIG. 5 depicts a front plan view of one version of a shaft clamp portion of the instrument adapter of FIG. 4;

FIG. 6 depicts a perspective view of the shaft clamp portion of FIG. 4 with a second clamp end of the shaft clamp portion in an open configuration;

FIG. 7 depicts a perspective view of another version of a shaft clamp portion of the instrument adapter of FIG. 4;

FIG. 8 depicts a front plan view of yet another version of a shaft clamp portion of the instrument adapter of FIG. 4;

FIG. 9 depicts a schematic perspective view of another version of an instrument adapter of the present disclosure secured to a carriage of a tool driver;

FIG. 10 depicts a flowchart of a first example of a workflow for utilizing the instrument adapters of the present disclosure with a secondary tool and a robotic surgical system; and

FIG. 11 depicts a flowchart of a second example of a workflow for utilizing the instrument adapters of the present disclosure with a secondary tool and a robotic surgical system.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Overview of an Example Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Example of Robotic System Table

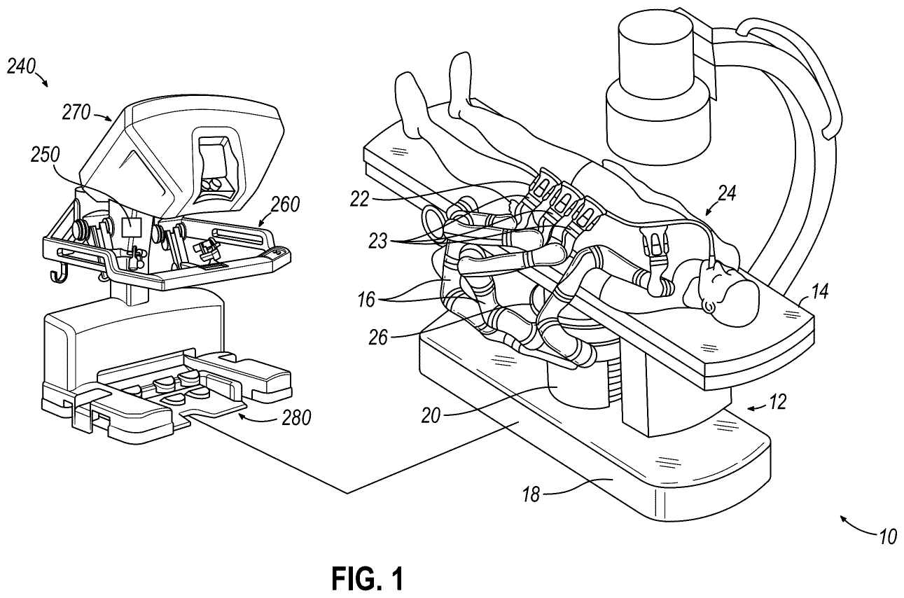

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) structurally supports platform (14) and provides a path for vertical translation of the carriages. In some versions, a table base may stow and store robotic arms (16) when not in use. Column (20) of the present example also includes a ring-shaped carriage (26), from which robotic arms (16) are based. A surgeon console (240) may be coupled with robotic surgical system (10).

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable locations. Tool drivers (22) are positioned at distal ends of robotic arms (16) in the present example. Tool drivers (22) are operable to manipulate one or more surgical instruments (24), as will be described in greater detail below.

Surgeon console (240) includes a control unit (250), a hand-control console (260), a display unit (270), and a foot control console (280). As will be described in greater detail below, surgeon console (240) may be utilized by a surgeon in order to view suitable data (e.g. visual images of the surgical site, current status of various system (10) components, etc.) and/or to manipulate robotic arms (16), tool drivers (22), and/or surgical instruments (24) during a surgical procedure.

### B. Example of a Robotic Arm, Tool Drive, and Tool

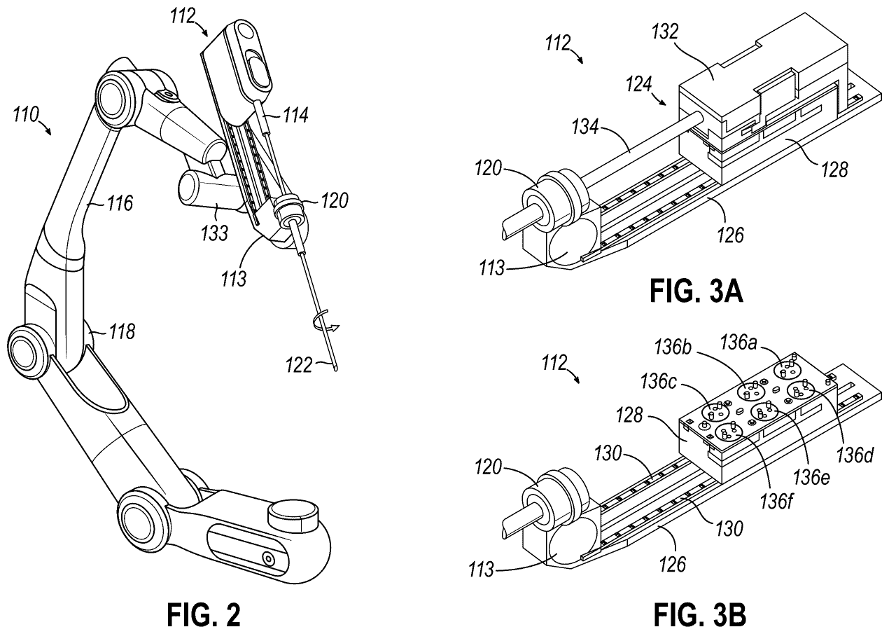

FIG. 2 shows an example of a robotic arm (110), a tool driver (112), and a surgical instrument (114), which may be incorporated into robotic surgical system (10) in place of robotic arm (16), tool driver (22), and surgical instrument (24) that are shown in FIG. 1. Additional examples of robotic arms, a tool drivers, and a surgical instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) may be attached to the distal end of robotic arm (110). Tool driver (112) includes a cannula (120) coupled to a distal end (113) of tool driver (112), to receive and guide surgical instrument (114). Surgical instrument (114) may include an endoscope, a laparoscope, a stapler, graspers, an ultrasonic instrument, an RF electrosurgical instrument, or any other suitable kind of instrument. Surgical instrument (114) may be inserted into the patient via cannula (120). A distal end of surgical instrument (114) includes an end effector (122). End effector (122) may be configured to interact with the patient (e.g., providing visualization, stapling, grasping, ultrasonic cutting and/or sealing, electrosurgical cutting, and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct a surgical instrument (114) from a desired point in space while allowing the clinician to move joints (118) into a clinically advantageous position away from the patient to create greater access, while avoiding collisions of robotic arms (110).

FIGS. 3A and 3B show tool driver (112) with and without a tool driver adapter (124), which may also be referred to as a smart tool base. As shown in FIGS. 3A and 3B, tool driver (112) may include a stage (126) and a carriage (128). Stage (126) includes longitudinal tracks (130). Carriage (128) may be slidingly engaged with longitudinal tracks (130). Stage (126) may be configured to couple to the distal end of robotic arm (110) such that articulation of robotic arm (110) positions and/or orients tool driver (112) in space. Surgical instrument (114) includes a tool driver adapter (124) at a proximal end and, as noted above, end effector (122) at a distal end. Tool driver adapter (124) includes a handle (132) and a shaft assembly (134) that extends distally from handle (132).

Carriage (128) may be configured to couple with tool driver adapter (124). Carriage (128) may drive a set of articulated movements of end effector (122) and/or otherwise actuate end effector (122), such as through a cable system or wires manipulated and controlled by actuated drives. Carriage (128) may include different configurations of actuated drives, including but not limited to motorized rotary axis drives. The plurality of rotary axis drives may be arranged in any suitable manner. As shown in FIG. 3B, carriage (128) of the present example includes six rotary drives (136a-f) arranged in two rows, extending longitudinally along the base of carriage (128). Rotary drives (136a-c) are arranged in a first row that may be longitudinally offset from a second row in which rotary drives (136d-f) are arranged. This staggered arrangement of rotary drives (136a-f) may reduce the width of carriage (128) and thereby provide a more compact form factor for tool driver (112). However, rotary drives (136a-f) may be provided in any other suitable arrangement. Moreover, any other suitable kind(s) of drive outputs may be provided by carriage (128), in addition to or in lieu of rotary drives (136a-f).

### II. Instrument Adapter for Use With Robotic Surgical Systems

### A. Laparoscopic Carriage Mounted Instrument Adapter

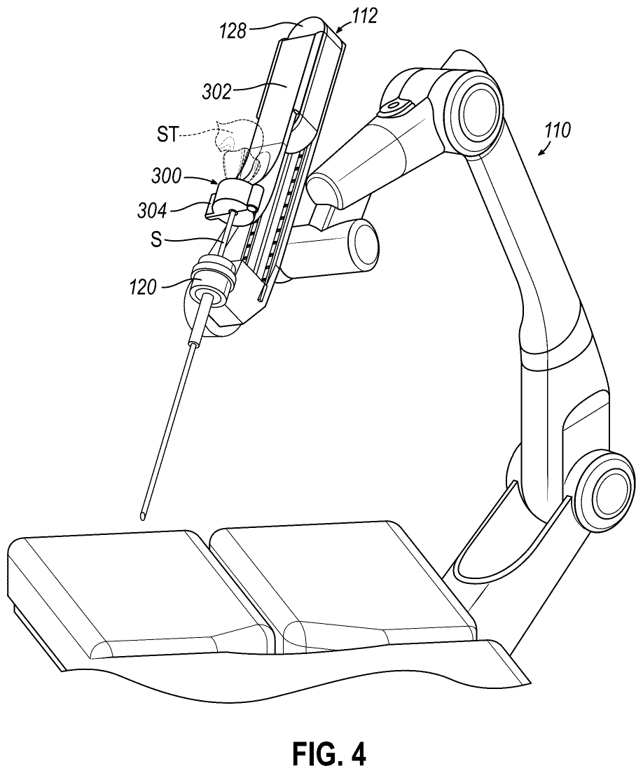

FIG. 4 shows an example version of an instrument adapter (300). Instrument adapter (300) may include a carriage mount portion (302) and a secondary tool clamp portion (304). As shown in FIG. 4, secondary tool clamp portion (304) is in the form of a shaft clamp portion (304). Although shown in FIG. 4 as being integral portions of adapter (300), it may also be contemplated that carriage mount portion (302) may contain an aperture into which shaft clamp portion (304) may be inserted into such that carriage mount portion (302) may be a separate element from shaft clamp portion (304). FIG. 4 also shows a shaft(S) of a secondary tool (ST) carried within shaft clamp portion (304) of instrument adapter (300).

In practice, carriage mount portion (302) may be receivable over carriage (128). In one or more versions, carriage mount portion (302) may snap into place over the periphery of carriage (128) or carriage mount portion (302) may contain rotary drives (not shown) which interact with rotary drives (136a-f) of carriage (128) to secure carriage mount portion (302) in position with carriage (128).

In one or more versions, adapter (300) may include an indicating system (not shown) which may indicate to robotic surgical system (10) that adapter (300) has been attached to carriage (128). Indicating system may indicate that robotic arm (110), that adapter (300) has been secured to, should go into Bedside Assist Mode. In one or more versions, Bedside Assist Mode sets system limitations and performance parameters, such as gravity compensation, on robotic arm (110) that adapter (300) has been secured to.

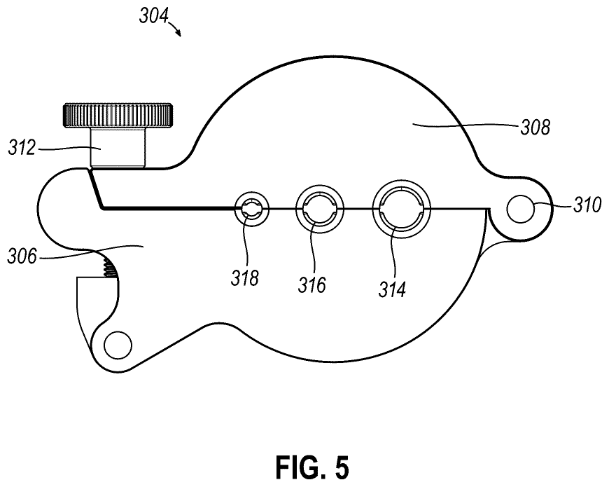

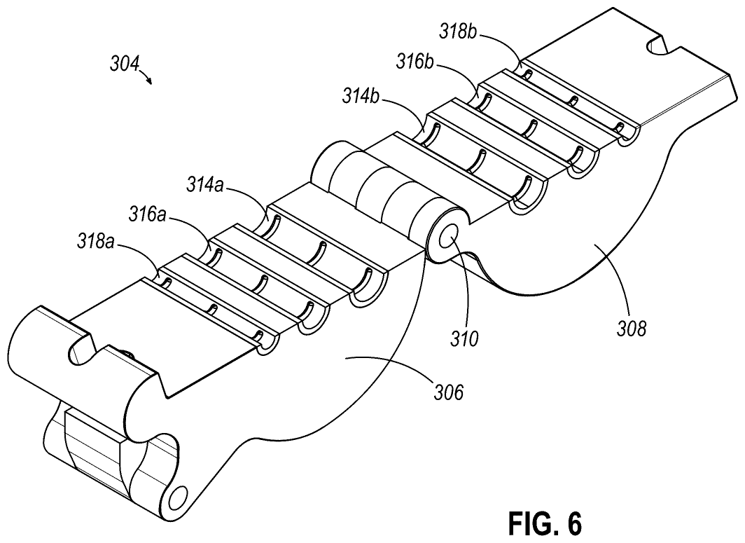

One embodiment of shaft clamp portion (304) is shown in FIGS. 5 and 6. Shaft clamp portion (304) includes a first clamp portion (306) and a second clamp portion (308). In one or more versions, first clamp portion (306) may be hingedly connected to second clamp portion (308) about hinge member (310). In one or more versions, second clamp portion (308) may be securable to first clamp portion (306) through a retainment device (312). In one or more versions, retainment device (312) can be a thumb screw, clamp, latch, slide tool, a magnetic latch, a toggle latch or a combination thereof.

In one or more versions, first clamp portion (306) may include a first half (314a) of a first tool aperture (314), a first half (316a) of a second tool aperture (316), and a first half (318a) of a third tool aperture (318) while second clamp portion (308) may include a second half (314b) of first tool aperture (314), a second half (316b) of second tool aperture (316), and a second half (318b) of third tool aperture (318). As can be seen in FIG. 5, first half (314a) mates with second half (314b) to form first tool aperture (314) when second clamp portion (308) may be secured to first clamp portion (306), first half (316a) mates with second half (316b) to form second tool aperture (316) when second clamp portion (308) may be secured to first clamp portion (306), and first half (318a) mates with second half (318b) to form third tool aperture (318) when second clamp portion (308) may be secured to first clamp portion (306). In one or more versions, a diameter of each tool aperture (314), (316), and (318) can be different to provide resistance and to hold different sized shafts of various secondary tools. Although shown with three tool apertures (314), (316), and (318), it may be contemplated that shaft clamp portion (304) can include more than three tool apertures or less than three tool apertures.

In one or more versions, tool apertures (314), (316), and (318) may be made from a medical grade elastomer such as silicone. In one or more versions, tool apertures (314), (316), and (318) may be a replaceable feature of shaft clamp portion (304).

In one or more versions, the selected tool aperture between (314), (316), and (318) may need to align with a trocar cannula centerline axis (not shown) which generally aligns with cannula (120) as seen in FIG. 4. In one or more versions, this alignment may be achieved by offsetting shaft clamp portion (304) from carriage mount portion (302) based on which tool aperture may be selected for use. In yet other versions, shaft clamp portion (304) may be movable in relation to carriage mount portion (302) to be able to slide clamp portion (304) in order to align the selected tool aperture with the trocar cannula centerline axis.

In use, retainment device (312) can be adjusted to allow for second clamp portion (308) to hingedly pivot away from first clamp end (306), as shown in FIG. 6. Once in this position, a secondary tool (ST) can be placed within one of the three tool apertures (314), (316), or (318), such as shown in FIG. 4. The shaft of secondary tool (ST) may be sized such that it may be placed within tool aperture (314), although, as discussed above, different secondary tools can be placed within whichever tool aperture (314), (316), or (318) provides the best fit for the secondary tool selected.

In one or more use cases, a sterile drape assembly (not shown) should be positioned between tool driver (112) and the secondary tool (ST) carried by instrument adapter (300). In one or more versions, the sterile drape assembly will include a drape portion, a tool sterile adapter portion, and a cannula sterile adapter portion. In one or more versions, drape portion includes a medical sheet that may be used to create the sterile field during surgery and other medical procedures; while the tool sterile adapter portion allows for securement of a surgical tool, such as surgical tool (114), or adapter (300) once the drape portion has been placed, and cannula sterile adapter portion allows for placement of a cannula, such as cannula (120), to distal end (113) of tool driver (112) once drape portion has been placed. In one or more versions, instrument adapter (300) may be combined with the tool sterile adapter portion of the sterile drape assembly such that placement of the tool sterile adapter portion will have the instrument adapter (300) positioned on a tool driver (112).

Once instrument adapter (300) and secondary tool (ST) are positioned on tool driver (112) of robotic arm (110), minimal robotic positional manipulation of secondary tool (ST) can occur via manipulation of robotic arm (110) at surgeon console (240). Additionally, positional manipulation of secondary tool (ST) can occur via manual manipulation of robotic arm (110) patient side by a clinician or a technician.

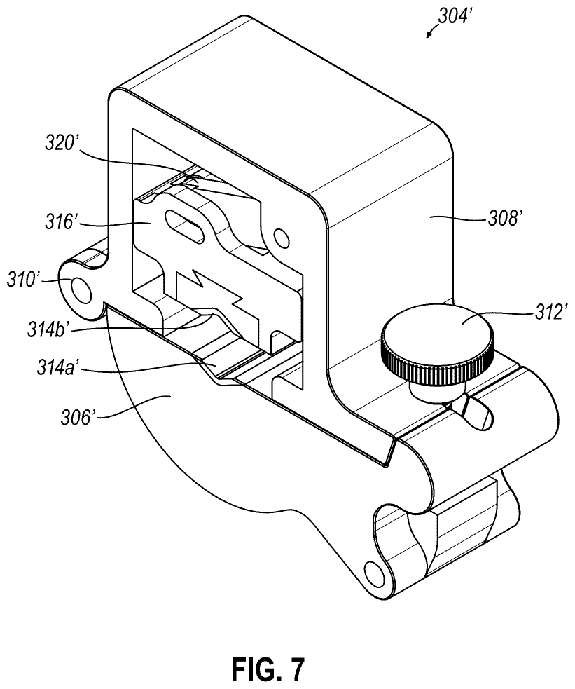

A second embodiment of shaft clamp portion (304′) is shown in FIG. 7. Shaft clamp portion (304′) includes a first clamp portion (306′) and a second clamp portion (308′). In one or more versions, first clamp portion (306′) may be hingedly connected to second clamp portion (308′) about hinge member (310′). In one or more versions, second clamp portion (308′) may be securable to first clamp portion (306′) through a retainment device (312′). In one or more versions, retainment device (312′) can be a thumb screw, clamp, latch, or a combination thereof.

In one or more versions, first clamp portion (306′) may include a first half of a tool aperture (314a′), while second clamp portion (308′) may include a tool press element (316′) that may include a second half of a tool aperture (314b′). Second clamp portion (308′) further may include one or more resilient elements (320′) which allow for movement of tool press element (316′) up and down within second clamp portion (308′). The moveable nature of tool press element (316′) allows for shaft clamp portion (304′) to fit different sized shafts of various secondary tools.

In use, a position of tool press element (316′) can be adjusted by adjustment of resilient elements (320′) to provide enough room between first half of tool aperture (314a′) and second half of tool aperture (314b′) to provide the best fit for the secondary tool selected. In one or more versions, first half of tool aperture (314a′) and second half of tool aperture (314b′) may be constructed to prevent damage to the secondary tool selected. In one or more versions, first half of tool aperture (314a′) and second half of tool aperture (314b′) may be constructed to minimize axial and rotational movement of the secondary tool selected. In one or more versions, first half of tool aperture (314a′) and second half of tool aperture (314b′) may be constructed from a medical grade elastomeric material such as silicone. Once the shaft of the secondary tool is placed between first half of tool aperture (314a′) and second half of tool aperture (314b′), tool press element (316′) can be adjusted into a position such that the shaft of secondary tool may be secured between first half of tool aperture (314a′) and second half of tool aperture (314b′).

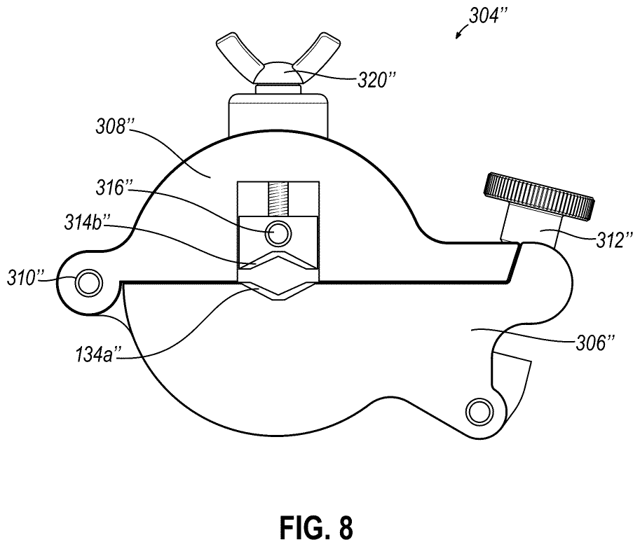

A third embodiment of shaft clamp portion (304″) is shown in FIG. 8. Shaft clamp portion (304″) includes a first clamp portion (306″), and a second clamp portion (508″). In one or more versions, first clamp portion (306″) may be hingedly connected to second clamp portion (308″) about hinge member (310″). In one or more versions, second clamp portion (308″) may be securable to first clamp portion (306″) through a retainment device (312″). In one or more versions, retainment device (312″) can be a thumb screw, clamp, latch, or a combination thereof.

In one or more versions, first clamp portion (306″) may include a first half of a tool aperture (314a″), while second clamp portion (308″) may include a tool press element (316″) that may include a second half of a tool aperture (314b″). Second clamp portion (308″) may also include a screw press element (320″) which may allow for movement up and down of tool press element (316″) within second clamp end (308″). The moveable nature of tool press element (316″) allows for shaft clamp portion (304″) to fit different sized shafts of various secondary tools.

In use, a position of tool press element (316″) can be adjusted by adjustment of screw press element (320″) to provide enough room between first half of tool aperture (314a″) and second half of tool aperture (314b″) to provide the best fit for the secondary tool selected. Once the shaft of the secondary tool is placed between first half of tool aperture (314a″) and second half of tool aperture (314b″), tool press element (316″) can be adjusted into a position such that the shaft of secondary tool may be secured between first half of tool aperture (314a″) and second half of tool aperture (314b″). In one or more versions, adjustment of screw press element (320″) is adjustable to allow for secondary tools with shaft diameters between about 3 mm and about 15 mm.

In yet other versions, screw press element (320″) may be replaced with a spring-based design such that tool press element (316″) may always be biased in a position against first half of tool aperture (314a″). In such a version, the spring compression of the spring-based design may be large enough to put enough pressure on the shaft of the secondary tool selected so as to minimize rotation and axial movement. In such a version, adjustment of the spring-based design would allow for secondary tools with shaft diameters between about 3 mm and about 15 mm.

### B. Laparoscopic Carriage Mounted Instrument Adapter With Clamping Wings

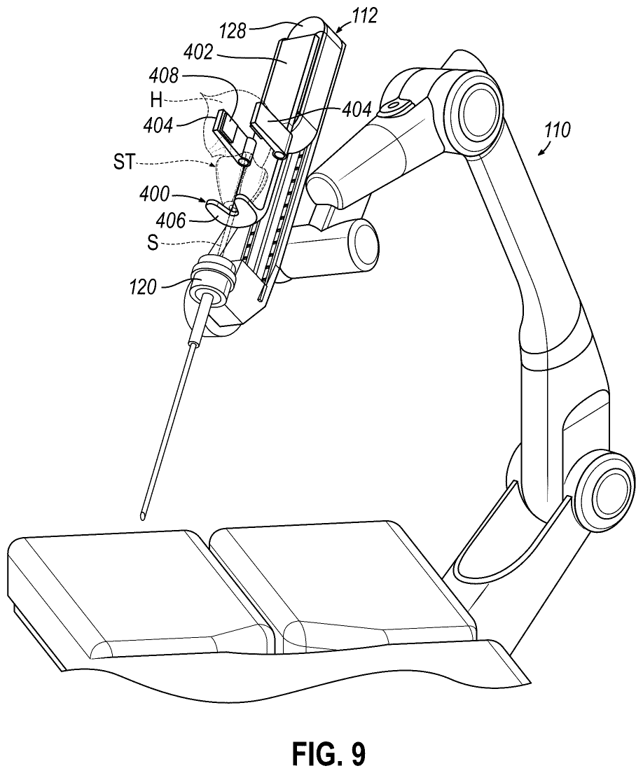

FIG. 9 shows an example version of an instrument adapter (400). Instrument adapter (400) may include a carriage mount portion (402), a secondary tool clamp portion (404), and an instrument shaft alignment portion (406). As shown in FIG. 9, secondary tool clamp portion (404) is in the form of a handle clamp portion (404). FIG. 9 also shows a handle H of a secondary tool (ST) carried within handle clamp portion (404) of instrument adapter (400). In one or more versions, handle clamp portion (404) includes a pair of spring-loaded clamp arms (408) that are operatively configured to provide resistance and hold a handle H of secondary tool (ST) when arms (408) are in a closed configuration. In one or more versions, each spring-loaded clamp arm of the pair of clamp arms (408) may include a conforming pad that directly contacts and forms to the shape of handle H of secondary tool ST to accommodate the position to contact handle H of secondary tool ST for the best possible retention of handle H of secondary tool ST. In one or more versions, conforming pad may be made from a medical grade elastomer such as silicone. As shown in FIG. 9, once handle H of secondary tool (ST) is loaded between clamp arms (408), a shaft(S) of secondary tool (ST) may be held in an aligned position by instrument shaft alignment portion (406).

In practice, carriage mount portion (402) may be receivable over carriage (128). In one or more versions, carriage mount portion (402) may snap into place over the periphery of carriage (128), or carriage mount portion (402) may contain rotary drives (not shown) which interact with rotary drives (136a-f) of carriage (128) to secure carriage mount portion (402) in position with carriage (128).

In one or more use cases, a sterile drape (not shown) should be positioned between tool driver (112) and the secondary tool carried by instrument adapter (400). In one or more use cases, a sterile drape assembly (not shown) should be positioned between tool driver (112) and the secondary tool (ST) carried by instrument adapter (400). In one or more versions, the sterile drape assembly will include a drape portion, a tool sterile adapter portion, and a cannula sterile adapter portion. In one or more versions, the drape portion includes a medical sheet that may be used to create the sterile field during surgery and other medical procedures; while the tool sterile adapter portion allows for securement of a surgical tool, such as surgical tool (114), or adapter (400) once the drape portion has been placed, and cannula sterile adapter portion allows for placement of a cannula, such as cannula (120), to distal end (113) of tool driver (112) once drape portion has been placed. In one or more versions, instrument adapter (400) may be combined with the tool sterile adapter portion of the sterile drape assembly such that placement of the tool sterile adapter portion will have the instrument adapter (300) positioned on a tool driver (112).

Once instrument adapter (400) and the secondary tool are positioned on tool driver (112) of robotic arm (110), minimal robotic positional manipulation of the secondary tool can occur via manipulation of robotic arm (110) at surgeon console (240). Additionally, positional manipulation of the secondary tool can occur via manual manipulation of robotic arm (110) patient side by a clinician or a technician.

In one or more versions, adapter (400) may include an indicating system (not shown) which will indicate to robotic surgical system (10) that adapter (400) has been attached to carriage (128). This indicating system may indicate that robotic arm (110), that adapter (400) has been secured to, should go into Bedside Assist Mode. In one or more versions, Bedside Assist Mode sets system limitations and performance parameters, such as gravity compensation, on the robotic arm (110) that adapter (400) has been secured to.

### C. Utilization of Universal Laparoscopic Carriage Mounted Instrument Adapter

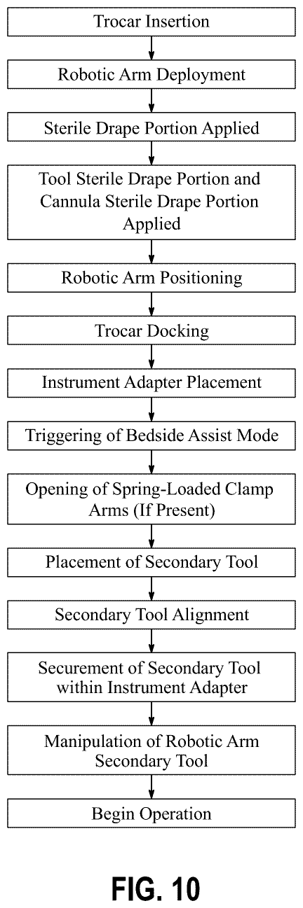

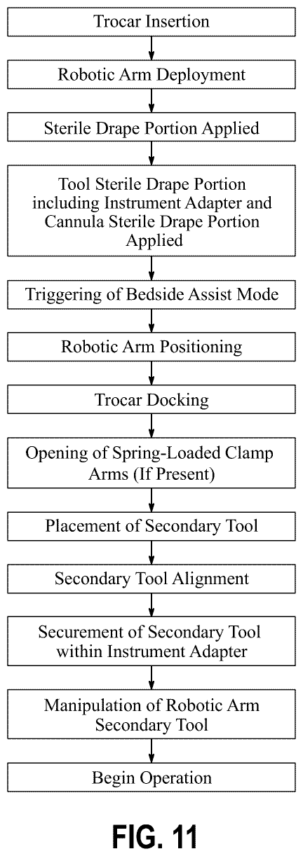

A first example of a workflow (500) for utilizing a secondary tool with one of the instrument adapters discussed above, such as instrument adapter (300) or (400) is shown in FIG. 10. In a first optional step (501), a trocar may be inserted near the targeted anatomy of a patient. In a second step (502), a robotic arm, such as robotic arm (110), may be deployed. In a third step (503), a sterile drape portion from a sterile drape assembly may be applied over robotic arm (110). In a fourth step (504), a tool sterile adapter portion and a cannula sterile adapter portion from a sterile drape assembly may be affixed to carriage (128) of tool driver (112) and distal end (113) of tool driver (112), respectively. In one or more versions, if step (501) does not take place first, then step (501) may also be done after step (504). In a fifth step (505), robotic arm (110) can be positioned either by a clinician working a surgeon console, such as surgeon console (240), or by manual maneuvering by a bedside clinician adjacent robotic arm (110). In a sixth step (506), the trocar placed step (501) can be docked to distal end (113) of tool driver (112), which now includes the cannula sterile adapter portion from the sterile drape assembly. In a seventh step (507), instrument adapter (300) or (400) may be positioned with the tool sterile adapter portion of the sterile drape assembly. In an eighth step (508), the placement of instrument adapter (300) or (400) triggers robotic arm (110) to go into Bedside Assist Mode. If instrument adapter placed in step (507) is instrument adapter (400), then in a ninth step (509), the pair of spring-loaded clamp arms (408) are opened. If instrument adapter placed in step (507) is instrument (300), then step (509) may be skipped.

In a tenth step (510), the secondary tool may be placed with in the trocar placed in step (501) and docked in step (506). In an eleventh step (511), the secondary tool may be aligned within the targeted anatomy as needed. In a twelfth step (512), the secondary tool may be secured to the instrument adapter. If the instrument adapter is instrument adapter (300), step (512) may include placing of the shaft from the secondary tool within shaft clamp portion (304, 304′, 304′) of instrument adapter (300). If the instrument adapter is instrument adapter (400), step (512) may include closing spring-loaded clamp arms (408) around the handle of the secondary tool. In a thirteenth step (513), robotic arm (110) and or the secondary tool may be manipulated as needed. The manipulation allowable within step (513) may be limited by robotic arm (110) being in Bedside Assist Mode, which occurred in step (508). In a fourteenth step (514), the operation may begin with the secondary tool being held in place by instrument adapter in Bedside Assist Mode as discussed above.

A second example of a workflow (600) for utilizing a secondary tool with one of the instrument adapters discussed above, such as instrument adapter (300) or (400) is shown in FIG. 11. Workflow (600) may be utilized in the instance wherein instrument adapter (300) or (400) have been combined with the tool sterile adapter portion of the sterile drape assembly. In a first step (601), a trocar may be inserted near the targeted anatomy of a patient. In a second step (602), a robotic arm, such as robotic arm (110), may be deployed. In a third step (603), a sterile drape portion from a sterile drape assembly may be applied over robotic arm (110). In a fourth step (604), a tool sterile adapter portion including instrument adapter (300) or (400) and a cannula sterile adapter portion from a sterile drape assembly may be affixed to carriage (128) of tool driver (112) and distal end (113) of tool driver (112), respectively. In a fifth step (605), the placement of tool sterile adapter portion including instrument adapter (300) or (400) triggers robotic arm (110) to go into a Bedside Assist Mode. In a sixth step (606), robotic arm (110) can be positioned either by a clinician working a surgeon console, such as surgeon console (240), or by manual maneuvering by a bedside clinician adjacent robotic arm (110).

In a seventh step (607), the trocar placed step (601) can be docked to distal end (113) of tool driver (112), which now includes the cannula sterile adapter portion from the sterile drape assembly. If tool sterile adapter portion placed in step (604) includes instrument adapter (400), then in an eighth step (608), the pair of spring-loaded clamp arms (408) are opened. If tool sterile adapter portion placed in step (604) includes instrument adapter instrument (300), then step (608) may be skipped. In a ninth step (609), the secondary tool may be placed with in the trocar placed in step (601) and docked in step (607). In a tenth step (610), the secondary tool may be aligned within the targeted anatomy as needed. In an eleventh step (611), the secondary tool may be secured to the instrument adapter. If the tool sterile adapter portion includes instrument adapter (300), step (611) may include placing of the shaft from the secondary tool within shaft clamp portion (304, 304′, 304′) of instrument adapter (300). If the tool sterile adapter portion includes instrument adapter (400), step (611) may include closing spring-loaded clamp arms (408) around the handle of the secondary tool. In a twelfth step (612), robotic arm (110) and or the secondary tool may be manipulated as needed. The manipulation allowable within step (612) may be limited by robotic arm (110) being in Bedside Assist Mode, which occurred in step (605). In a thirteenth step (613), the operation may begin with the secondary tool being held in place by instrument adapter in Bedside Assist Mode as discussed above.

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A robotic surgical system, comprising: a robotic arm including a distal end; a tool driver operatively coupled with the distal end of the robotic arm and including a carriage at a proximal end; a control unit; and an instrument adapter comprising: a carriage mount portion, and a secondary tool clamp portion, wherein the carriage mount portion is operatively receivable on the carriage at the proximal end of the tool driver, and wherein the secondary tool clamp portion is operatively configured to provide resistance and hold a secondary tool in position.

### EXAMPLE 2

The robotic surgical system of any one or more the preceding Examples, wherein the secondary tool clamp portion includes a shaft clamp portion.

### EXAMPLE 3

The robotic surgical system of any one or more the preceding Examples, wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

### EXAMPLE 4

The robotic surgical system of any one or more the preceding Examples, wherein the first clamp end is hingedly connected to the second clamp end about a hinge member and wherein the first clamp end is securable to the second clamp end through a retainment device.

### EXAMPLE 5

The robotic surgical system of any one or more the preceding Examples, wherein the first clamp end includes one or more first halves of one or more tool apertures, wherein the second clamp end includes one or more second halves of the one or more tool apertures, and wherein the one or more first halves align with the one or more second halves when the first clamp end is secured to the second clamp end to form the one or more tool apertures.

### EXAMPLE 6

The robotic surgical system of any one or more the preceding Examples, wherein the one or more tool apertures are operably configured to receive a shaft of the secondary tool when the first clamp end is secured to the second clamp end.

### EXAMPLE 7

The robotic surgical system of any one or more the preceding Examples, wherein the first clamp end includes a first half of a tool aperture and wherein the second clamp end includes a tool press element having a second half of the tool aperture

### EXAMPLE 8

The robotic surgical system of any one or more the preceding Examples, wherein the tool press element further includes one or more resilient elements which allow for up and down movement of the tool press element within the second clamp end.

### EXAMPLE 9

The robotic surgical system of any one or more the preceding Examples, wherein movement of the tool press element by the resilient elements within the second clamp end allows for the instrument adapter to be operatively configured to receive secondary tools with varying shaft sizes when the first clamp end is secured to the second clamp end.

### EXAMPLE 10

The robotic surgical system of any one or more the preceding Examples, wherein the second clamp end further includes a screw press element which allows for up and down movement of the tool press element within the second clamp end.

### EXAMPLE 11

The robotic surgical system of any one or more the preceding Examples, wherein movement of the tool press element by the screw press element allows for the instrument adapter to be operatively configured to receive secondary tools with varying shaft sizes when the first clamp end is secured to the second clamp end.

### EXAMPLE 12

The robotic surgical system of any one or more the preceding Examples, wherein the secondary tool clamp portion includes a handle clamp portion.

### EXAMPLE 13

The robotic surgical system of any one or more the preceding Examples, wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

### EXAMPLE 14

The robotic surgical system of any one or more the preceding Examples, wherein each spring-loaded clamp arm of the pair of spring-loaded clamp arms include a conforming pad that is operatively configured to directly contact and form to a shape of the handle of the secondary tool.

### EXAMPLE 15

An instrument adapter for use with a tool driver of a robotic surgical system comprising: a carriage mount portion, and a secondary tool clamp portion, wherein the carriage mount portion is operatively receivable on a carriage at a proximal end of the tool driver, and wherein the secondary tool clamp portion is operatively configured to provide resistance and hold a secondary tool in position

### EXAMPLE 16

The robotic surgical system of Example 15, wherein the secondary tool clamp portion includes a shaft clamp portion, and wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

### EXAMPLE 17

The robotic surgical system of Example 15, wherein the secondary tool clamp portion includes a handle clamp portion, and wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

### EXAMPLE 18

A method of utilizing an instrument adapter and sterile drape assembly with a robotic surgical system to hold a secondary tool, the robotic surgical system including a robotic arm including a distal end, a tool driver operatively coupled with the distal end of the robotic arm and including a carriage at a proximal end and a distal end, and a control unit, the instrument adapter including a carriage mount portion and a secondary tool clamp portion, and the sterile drape assembly including a sterile drape portion, a tool sterile adapter portion, and a cannula sterile adapter portion, the method comprising: applying the sterile drape portion of the sterile drape assembly over the robotic arm of the robotic surgical system; affixing the tool sterile adapter portion of the sterile drape assembly to the carriage of the tool driver and the cannula sterile adapter portion to the distal end of the tool driver; securing the carriage mount portion of the instrument adapter onto the tool sterile adapter portion of the sterile drape assembly; and securing the secondary tool with the secondary tool clamp portion of the instrument adapter, thereby utilizing the instrument adapter with the robotic surgical system.

### EXAMPLE 19

The method of Example 18, wherein the secondary tool clamp portion includes a shaft clamp portion, and wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

### EXAMPLE 20

The method of Example 18, wherein the secondary tool clamp portion includes a handle clamp portion, and wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

### EXAMPLE 21

The method of Example 18, wherein the instrument adapter is an integral part of the tool sterile adapter portion of the sterile drape assembly such that the step of affixing the tool sterile adapter portion of the sterile drape assembly and the step of securing the carriage mount portion of the instrument adapter occurs in one simultaneous step.

### IV. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of uterine manipulator instruments, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A robotic surgical system, comprising:
(a) a robotic arm including a distal end;
(b) a tool driver operatively coupled with the distal end of the robotic arm and including a carriage at a proximal end;
(c) a control unit; and
(d) an instrument adapter comprising:
(i) a carriage mount portion, and
(ii) a secondary tool clamp portion,
wherein the carriage mount portion is operatively receivable on the carriage at the proximal end of the tool driver, and wherein the secondary tool clamp portion is operatively configured to provide resistance and hold a secondary tool in position.

2. The robotic surgical system of claim 1, wherein the secondary tool clamp portion includes a shaft clamp portion.

3. The robotic surgical system of claim 2, wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

4. The robotic surgical system of claim 3, wherein the first clamp end is hingedly connected to the second clamp end about a hinge member and wherein the first clamp end is securable to the second clamp end through a retainment device.

5. The robotic surgical system of claim 3, wherein the first clamp end includes one or more first halves of one or more tool apertures, wherein the second clamp end includes one or more second halves of the one or more tool apertures, and wherein the one or more first halves align with the one or more second halves when the first clamp end is secured to the second clamp end to form the one or more tool apertures.

6. The robotic surgical system of claim 5, wherein the one or more tool apertures are operably configured to receive a shaft of the secondary tool when the first clamp end is secured to the second clamp end.

7. The robotic surgical system of claim 3, wherein the first clamp end includes a first half of a tool aperture and wherein the second clamp end includes a tool press element having a second half of the tool aperture.

8. The robotic surgical system of claim 7, wherein the tool press element further includes one or more resilient elements which allow for up and down movement of the tool press element within the second clamp end.

9. The robotic surgical system of claim 8, wherein movement of the tool press element by the resilient elements within the second clamp end allows for the instrument adapter to be operatively configured to receive secondary tools with varying shaft sizes when the first clamp end is secured to the second clamp end.

10. The robotic surgical system of claim 7, wherein the second clamp end further includes a screw press element which allows for up and down movement of the tool press element within the second clamp end; wherein movement of the tool press element by the screw press element allows for the instrument adapter to be operatively configured to receive secondary tools with varying shaft sizes when the first clamp end is secured to the second clamp end.

11. The robotic surgical system of claim 1, wherein the secondary tool clamp portion includes a handle clamp portion.

12. The robotic surgical system of claim 11, wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

13. The robotic surgical system of claim 12, wherein each spring-loaded clamp arm of the pair of spring-loaded clamp arms include a conforming pad that is operatively configured to directly contact and form to a shape of the handle of the secondary tool.

14. An instrument adapter for use with a tool driver of a robotic surgical system comprising:
(i) a carriage mount portion, and
(ii) a secondary tool clamp portion,
wherein the carriage mount portion is operatively receivable on a carriage at a proximal end of the tool driver, and wherein the secondary tool clamp portion is operatively configured to provide resistance and hold a secondary tool in position.

15. The instrument adapter of claim 14, wherein the secondary tool clamp portion includes a shaft clamp portion, and wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

16. The instrument adapter of claim 14, wherein the secondary tool clamp portion includes a handle clamp portion, and wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

17. A method of utilizing an instrument adapter and sterile drape assembly with a robotic surgical system to hold a secondary tool, the robotic surgical system including a robotic arm including a distal end, a tool driver operatively coupled with the distal end of the robotic arm and including a carriage at a proximal end and a distal end, and a control unit, the instrument adapter including a carriage mount portion and a secondary tool clamp portion, and the sterile drape assembly including a sterile drape portion, a tool sterile adapter portion, and a cannula sterile adapter portion, the method comprising:
(a) applying the sterile drape portion of the sterile drape assembly over the robotic arm of the robotic surgical system;
(b) affixing the tool sterile adapter portion of the sterile drape assembly to the carriage of the tool driver and the cannula sterile adapter portion to the distal end of the tool driver;
(c) securing the carriage mount portion of the instrument adapter onto the tool sterile adapter portion of the sterile drape assembly; and
(d) securing the secondary tool with the secondary tool clamp portion of the instrument adapter, thereby utilizing the instrument adapter with the robotic surgical system.

18. The method of claim 17, wherein the secondary tool clamp portion includes a shaft clamp portion, and wherein the shaft clamp portion includes a first clamp end a second clamp end hindgedly connected and securable to the first clamp end and wherein the first clamp end and the second clamp end are operatively configured to provide resistance and hold a shaft of the secondary tool in position when the first clamp end is secured to the second clamp end.

19. The method of claim 17, wherein the secondary tool clamp portion includes a handle clamp portion, and wherein the handle clamp portion includes a pair of spring-loaded clamp arms and wherein the pair of spring-loaded clamp arms are operatively configured to provide resistance and hold a handle of the secondary tool in position when the pair of spring-loaded clamp arms are in a closed configuration.

20. The method of claim 17, wherein the instrument adapter is an integral part of the tool sterile adapter portion of the sterile drape assembly such that the step of affixing the tool sterile adapter portion of the sterile drape assembly and the step of securing the carriage mount portion of the instrument adapter occurs in one simultaneous step.
